(19) United States
(12) Patent Application Publication
(10) Pub. No.: US 2011/0257202 A1
(43) Pub. Date: Oct. 20, 2011

(54) Therapeutic Compositions and Methods for Treating Chronic Kidney Disease Associated with a Metabolic Imbalance

(76) Inventors: Richard J Johnson, Centennial, CO (US); Takahiko Nakagawa, Greenwood Village, CO (US)

(21) Appl. No.: 13/124,978

(22) PCT Filed: Oct. 19, 2009
(86) PCT No.: PCT/US09/61157

§ 371 Date: Jul. 8, 2011

Related U.S. Application Data
(60) Provisional application No. 61/106,602, filed on Oct. 19, 2008.

(51) Int. Cl. A61K 31/519; A61P 3/10; A61K 31/195; A61P 13/12; A61K 31/4406; A61K 31/426

(52) U.S. Cl. 514/262.1; 514/355; 514/365; 514/565

(57) ABSTRACT

Disclosed herein are compositions and methods for treating chronic kidney disease and/or a metabolic imbalance. Specifically exemplified herein are methods involving the coadministration of a RAS inhibitor with a conjunctive agent that improves endothelial NO or endothelial function. Also disclosed are methods of treating a patient exhibiting symptoms of a stage of chronic kidney disease and at least one symptom of a metabolic imbalance, such as one or more diagnostic criteria of the metabolic syndrome.

### RELATED APPLICATIONS

This application claims priority to U.S. Ser. No. 61/106,602, filed Oct. 19, 2008, which is incorporated herein in its entirety.

### BACKGROUND AND INTRODUCTION OF INVENTION

Diabetes currently affects 8% of the US population and the prevalence is increasing. One of most significant complications of diabetes is the development of nephropathy, which can progress to end stage renal disease requiring dialysis or transplantation. Currently angiotensin converting enzyme inhibitor (ACEI) and angiotensin receptor blockers (ARBs) are favored as treatments for diabetic nephropathy and the benefits are thought to be in part independent of their blood pressure lowering effects ¹⁻⁴. However, recent studies have suggested that ACE inhibitors and ARBs are not effective in slowing the development of type 1 diabetic nephropathy despite having benefit in preventing retinopathy⁵. Other groups have also reported that ACE inhibitors are not effective as originally postulated in type 2 diabetes with overt nephropathy ⁶⁻⁸. These observations are in contrast to the well known protective effect of ACE inhibitors and ARBs in the prevention of nephropathy in diabetic rats^{9, 10}.

The mechanism accounting for the lack of effect of ACE inhibitors in diabetic nephropathy is not known. However, some patients with diabetic nephropathy who are receiving ACE inhibitors or ARBs develop a paradoxical rise in aldosterone levels (termed “aldosterone breakthrough”) ^{11, 12 13}. The addition of aldosterone inhibitors with the use of ACE inhibitors has been suggested¹⁴, but these treatments are often limited by hyperkalemia, which is common when both treatments are combined.

The present invention is based on the inventors discovery that a lack of endothelial nitric oxide is underlying factor causing unresponsiveness of ACE inhibitors and/or ARBs in Diabetic Nephropathy. The invention presented provides a new means for increasing the efficacy of ACE inhibitors and ARBs in the prevention and treatment of diabetic nephropathy. Specifically, it is based on a series of discoveries that have revealed the mechanism for ACE inhibitor and ARB unresponsiveness in the diabetic state.

The development of any new therapeutic agent is often initially based on the efficacy of these agents on animal models of the targeted disease. In this regard, numerous models of diabetic nephropathy have been reported in rats and mice. However, until recently none of these models resembled human diabetic nephropathy. While models of type1 diabetes (such as the streptozotocin-induced diabetic rat) and type 2 diabetes (db db mouse) are well known, these models are associated with only mild proteinuria and early changes of diabetic renal disease such as the expansion of the mesangium and basement membrane thickening. Importantly, these models do not develop clinical manifestations (nephrotic proteinuria, progressive loss of glomerular filtration rate, GFR) or histologic lesions (mesangiolysis, mesangial nodules, vascular lesions or tubulointerstitial disease) that is observed in human diabetic nephropathy. The lack of a good model of human diabetic nephropathy has thwarted our understanding of the pathogenesis of the disease and also has made it difficult to test new therapies. Indeed, concern of the lack of a good model of diabetic nephropathy led the NIH to create a consortium with the specific goal of developing such an animal model¹⁵.

The inventors¹⁶⁻¹⁹ (see also²⁰) recently developed an animal model that closely resembles human diabetic nephropathy by using mice that are deficient in endothelial nitric oxide synthase and hence are unable to produce endothelial nitric oxide. It has been shown that when diabetes is induced in these mice with streptozotocin, that they develop all aspects of human diabetic nephropathy, including clinical manifestations (nephrotic proteinuria, progressive renal failure and early mortality), histologic manifestations (including mesangial expansion, nodules and mesangiolysis, podocyte abnormalities, vascular lesions, tubulointerstitial disease) and molecular changes (increases in TGF-β and VEGF expression). To the inventors' knowledge this is also the first diabetic model that develops both retinopathy and nephropathy spontaneously. Importantly, renal disease in these mice can be prevented with insulin²¹ and with effective blood pressure lowering¹⁸, similar to that reported in humans.

It had originally been expected that ACE inhibitors and ARBs would be effective in this model of diabetic nephropathy as it has been shown in other models⁹. Indeed, it was again documented that ACE inhibitors and ARBs could block the development of diabetic nephropathy in wild type mice injected with streptozotocin. However, to the inventors' surprise, this same treatment was not effective in diabetic eNOSKO mice.

### BRIEF DESCRIPTION OF THE DRAWINGS

FIG. 1 represents a graph showing that ACE inhibitors are effective in lowering blood pressure in wild type diabetic (DM) mice. Shown in FIG. 1 is the effect of no treatment (No TX), the ACE inhibitor enalapril (DM enalapril), and the ARB telmisartan (DM telmisartan). Both the ACE inhibitor and ARB can significantly lower blood pressure (BP) in wild type diabetic mice.

FIG. 2 ACE inhibitors (enalapril) and ARBs (telmisartan) also improve mesangial expansion (A) and glomerular type IV collagen deposition (B) in wild type diabetic (DM) mice. These are considered early changes of diabetic nephropathy.

FIG. 3 represents a graph showing that ACE inhibitors and ARBs are Less Effective at Lowering Blood Pressure in Diabetic Mice lacking Endothelial NO. 3(A) shows the effect of ACE inhibitors and ARBs on blood pressure in NonDM eNOSKO mice and 3(B) shows the effect ACE inhibitors and ARBs on blood pressure in DM eNOSKO mice.

FIG. 4 ACE inhibitors and ARBs are not effective at preventing diabetic nephropathy in eNOSKO mice. DM caused mesangial expansion in DM eNOSKO mice (B), which was not prevented by enalapril (C) or telmisartan (D). DM eNOSKO mice also exhibited mesangiolysis with glomerular capillary microaneurysms (E) and with augmented deposition of extracellular matrix (F). Neither enalapril (G) nor telmisartan (H) treatment prevented these advanced lesions. Bar: 20 μm. Quantitative analysis of mesangial expansion (M) confirmed a beneficial effect of ACE inhibitor (black bar) and ARB (grey bar) in wild type diabetic mice but not diabetic eNOSKO mice. Bar: 20 μm. White bar=no treatment; Black bar=enalapril; Gray bar=telmisartan. Data are shown as means and

FIG. 5 is a table showing the lack of effect of ACE inhibitors and ARBs on blood pressure and renal function in diabetic nephropathy in eNOSKO mice. ACE inhibitors and ARBs are effective at lowering blood pressure and proteinuria in wild type diabetic mice, and are also effective at lowering blood pressure in nondiabetic eNOSKO mice. However, ACE inhibitors are ineffective and ARAB are only minimally effective at lowering blood pressure and do not significantly reduce proteinuria in diabetic eNOSKO mice at 10 weeks.

FIG. 6 represents graphs showing Serum Aldosterone is suppressed in diabetic wild type mice (A) but is not suppressed by ACE inhibitor or ARB treatment in diabetic eNOS KO mice (B).

FIG. 7 is an immunohistograph of a kidney of a Diabetic eNOSKO mouse showing an increase in aldosterone in its glomeruli. Diabetes was induced with streptozotocin in eNOSKO mice, which were treated with enalapril (10 mg/kg BW/day; black bar) or telmisartan (2 mg/kg BW/day; gray bar) for 4 weeks (from at 6 weeks to 10 weeks)

FIG. 8 is a graph showing that supply of a Nitric Oxide Mimetic (Nitrite) can correct the blood pressure abnormality in the eNOSKO mouse.

FIG. 9 are immunohistographs and graph showing that uric acid causes oxidative stress in human aortic endothelial cells. The green fluorescence is a measure of a marker for oxidative stress using the peroxide-sensitive probe 2′7′-dichlorofluorescein diacetate (DCF-DA) dye (FIG. 9A). On the right is the quantitation of oxidative stress, with control in open bar, uric acid (12 mg/dl) in the black bar, and uric acid with apocynin (an inhibitor of NADPH oxidase-induced oxidants) in the right bar (FIG. 9B).

FIG. 10 represents a graph showing that uric acid causes a reduction in endothelial NO levels. Shown is the effect of various doses of uric acid on endothelial NO (measured by fluorescence of DAF in porcine aortic endothelial cells. From Khosla et al³⁰

FIG. 11 is a bar graph showing that uric acid reduces ATP levels in human aortic endothelial cells. Uric acid (3.5 mg/dl, 7 mg/dl and 12 mg/dl) was included in the media for 48 hours. No alterations in cell viability (trypan blue exclusion) were observed.

FIG. 12 represents images and graph showing that uric acid reduces mitochondria number in human aortic endothelial cells. Mitochondria numbers were measured with Mitotracker Orange in control cells (FIG. 12A) and UA treated cells (FIG. 12B). Shown in FIG. 12C is the quantified effect of uric acid 12 mg/dl on mitochondria density at 48 hours. No effects on cell viability was observed.

### DETAILED DESCRIPTION

Recent research conducted by the inventors reveals that agents that improve endothelial dysfunction and endothelial NO should be of therapeutic benefit for a metabolic imbalance, in diabetic nephropathy, in particular. These agents act in part by the improvement of mitochondrial function. Agents contemplated for use in accordance with the invention include, but are not limited to agents that lower uric acid (UALA), such as xanthine oxidase inhibitors (febuxostat, allopurinol), uricosurics (benziodarone, benzbromarone, probenecid), uricase derivatives (Rabsuricase, Pegylated uricase) and gene based therapies (uricase overexpression) or blockade of URAT-1 (the transporter we have identified on the vascular endothelial cell); NO mimetics such as long acting nitrates, L-arginine and Nicorandil; and antioxidants such as ascorbate, N acetyl cystein, epigallocatechin gallate and epicatechin.

It is also asserted that agents that stimulate NO will have direct synergy with ACE inhibitors, or other agents targeting the renin-angiotensin pathway, in the treatment of diabetic nephropathy. The combination will potentiate the effects of ACE inhibitors on blood pressure, renal structure, and renal function.

According to another embodiment, the subject invention pertains to a conjunctive therapy for treating metabolic imbalance and/or treating and/or preventing diabetic nephropathy, that comprises administering a therapeutically effective amount of a “RAS inhibitor”, i.e., an agent that targets the Renin-angiotensin pathway, including but not limited to ACE inhibitors renin inhibitor or angiotensin receptor blockers, and co-administering a therapeutically effective amount of a “conjunction agent”, including but not limited to, agents that lower uric acid (UALA), such as xanthine oxidase inhibitors (febuxostat, allopurinol), uricosurics (benziodarone, benzbromarone, probenecid), uricase derivatives (Rabsuricase, Pegylated uricase) and gene based therapies (uricase overexpression) or blockade of URAT-1 (the transporter we have identified on the vascular endothelial cell); NO mimetics such as long acting nitrates, L-arginine and Nicorandil; and antioxidants such as ascorbate, N acetyl cystein, lipoic acid, vitamin e, epigallocatechin gallate. With respect to either RAS inhibitors or conjunctive agents described herein, it should be noted that pharmaceutically acceptable salts of such agents also may be used. Any reference to a RAS inhibitor or conjunctive agent in the claims is construed to include, either optionally or additionally, the corresponding pharmaceutically acceptable salt of such agent.

In a particular embodiment, the conjunctive therapy comprises treating or preventing diabetic nephropathy by administering a composition comprising a RAS inhibitor and a conjunctive agent, or pharmaceutically acceptable salts thereof, as the primary active components. In a more specific embodiment, the method for treating or preventing diabetic nephropathy comprises administering an ACE inhibitor and a NO mimetic. In an even more specific embodiment, the NO mimetic is nicorandil.

A patient in need is one that is exhibiting symptoms of one of the classic stages of chronic kidney disease (CKD) as defined by the National Kidney Foundation and/or is exhibiting symptoms of a metabolic imbalance. In a more specific embodiment, the patient in need is one experiencing one of the classic stages of CKD and is exhibiting at least two characteristics of the metabolic syndrome.

In certain aspects of the invention, the metabolic imbalance is selected from the group consisting of: diabetes mellitus, gestational diabetes, genetic defects of β-cell function, genetic defects in insulin action, diseases of the exocrine pancreas, endocrinopathies, drug or chemical-induced, infections, other genetic syndromes associated with diabetes, a pre-diabetic state, and metabolic syndrome. In one aspect, the metabolic imbalance is diabetes mellitus, including type I and/or type II.

According to another aspect, the metabolic imbalance is the metabolic syndrome. In one aspect, treating metabolic syndrome comprises treating one or more diagnostic criteria. A patient exhibiting symptoms of the metabolic syndrome includes a patient exhibiting two or more of the following diagnostic criteria:

Elevated waist circumference:

Men—Equal to or greater than 40 inches (102 cm)

Women—Equal to or greater than 35 inches (88 cm)

Elevated triglycerides:

Equal to or greater than 150 mg/dL

Reduced HDL (“good”) cholesterol:

Men—Less than 40 mg/dL

Women—Less than 50 mg/dL

Elevated blood pressure:

Equal to or greater than 130/85 mm Hg

Elevated fasting glucose:

Equal to or greater than 100 mg/dL

In still another embodiment, the metabolic imbalance is a pre-diabetic state pertaining to an impaired fasting glucose level (equal to or greater than 100 mg/dL) or glucose intolerance (greater than 140 mg/dL two hours post premeasured glucose drink).

Chronic Kidney Disease stages pertain to the following:

CKD stage 1: normal or increased glomerular filtration rate (GFR); some evidence of kidney damage reflected by microalbuminuria,/proteinuria, hematuria or histologic changes.

CKD stage 2: mild decrease in GFR (defined as 89-60 ml/min/1.73 m2) as defined by MDRD GFR.

CKD stage 3 as moderate decrease in GFR (59-30 ml/min/1.73 m2) as defined by MDRD GFR.

CKD stage 4 as severe decrease in GFR (29-15 ml.min/1.73 m2)

The preceding descriptions represent clinically useful stages of chronic kidney disease that are readily discernable to the clinician and are detailed in National Kidney foundation: K/DOQI kidney disease outcome quality initiative. Am J Kidney Dis 2002; 39 (Suppl)1):S1-S266.

In another embodiment, a patient exhibiting symptoms of chronic kidney disease and a diagnostic criteria of the metabolic syndrome is treated.

ACE inhibitors have been touted as the best treatment of diabetic nephropathy—however, recent studies suggest they may be uniquely bad in diabetic nephropathy. The inventors have likely discovered the reason, and also the solution. In other words, if endothelial dysfunction can be improved, then ACE inhibitors will work much better.

In a further embodiment, therapeutic composition embodiments can be formulated in accordance via conventional procedures. Such a formulation can be produced usually by mixing/kneading active components, RAS inhibitor and conjunctive agent, with non-active additives such as an excipient, diluent and carrier. In this specification, a parenteral administration means to include subcutaneous injection, intravenous injection, intramuscular injection, intraperitoneal injection or dripping infusion and the like. A formulation for injection such as aseptic aqueous suspension or oily suspension for injection can be produced using a suitable dispersing agent or wetting agent and a suspending agent by a method known in the art. Such an aseptic formulation for injection may be an aseptic injectable solution or suspension in a diluent or solvent which can be non-toxic and administered parenterally, including an aqueous solution. An acceptable vehicle or solvent which can be employed may, for example, be water, Ringer's solution, isotonic saline and the like. An aseptic non-volatile oil can also be used usually as a solvent or suspending medium. For such purpose, any non-volatile oil or fatty acid can be employed, including naturally occurring or synthetic or semi-synthetic fatty oil or fatty acid, as well as naturally occurring or synthetic or semi-synthetic mono- or di- or tri-glycerides.

A suitable base (e.g. polymer of butyric acid, polymer of glycolic acid, copolymer of butyric acid and glycolic acid, mixture of a polymer of butyric acid and a polymer of glycolic acid, polyglycerol fatty acid ester and the like) may be combined to form a sustained release formulation.

In another embodiment, a solid dosage form for oral administration may, for example, be a powder, granule, tablet, pill, capsule and the like, as described above. The formulation of such a dosage form can be produced by mixing and/or kneading active compounds, RAS inhibitor or conjunctive agent, with at least one of the non-active additives, such as sucrose, milk sugar (lactose), cellulosic saccharide, mannitol (D-mannitol), maltitol, dextran, starches (e.g., corn starch), microcrystalline cellulose, agar, alginates, chitins, chitosans, pectins, tragacanth gums, gum arabic, gelatins, collagens, casein, albumin, synthetic or semi-synthetic polymers or glycerides. Such a dosage form can further contain additives as usual, including inert diluents, lubricants such as magnesium stearate, preservatives such as parabens and sorbic acid, antioxidants such as ascorbic acid, α-tocopherol and cysteine, disintegrants (e.g., croscarmellose sodium), binder (e.g., hydroxypropyl cellulose), thickening agents, buffering agents, sweeteners, flavoring agents, perfumes and the like. A tablet and pill may further be enteric-coated. An oral liquid formulation may, for example, be a pharmaceutically acceptable emulsion, syrup, elixir, suspension, solution and the like, which may contain a pharmaceutically customary inert diluent such as water and if desired, additives. Such an oral liquid formulation can be produced by mixing an active ingredient, inert diluent and other additives if necessary in accordance with a customary method. An oral formulation usually contain about 0.01 to 99% by weight, preferably about 0.1 to 90% by weight, usually about 0.5 to 50% by weight of an inventive active compound, although the amount may vary depending on the dosage form.

In an alternative embodiment, a suppository for rectal administration can be produced by mixing active components with a suitable non-irritating excipient which is solid at ambient temperature but becomes liquid at the temperature in an intestinal tract to melt in rectum whereby releasing the active ingredient, such as cocoa butter and polyethylene glycols.

The dose in a certain patient is determined considering the age, body weight, general condition, sex, diet, administration time, administration mode, excretion rate, drug combination, degree of the disease treated currently as well as other factors.

A diabetic nephropathy therapeutic composition of the present invention has a low toxicity and can be used safely, and its daily dose varies depending on the condition and body weight of the patient, the type of the compound and the administration route and, for example, when used as a prophylactic and therapeutic against diabetic nephropathy, it may be about 1 to 500 mg, typically about 10 to 200 mg as an active ingredient [I] in an oral formulation, and about 0.1 to 100 mg, typically about 1 to 50 mg, usually about 1 to 20 mg as an active ingredient [I] in a parenteral formulation for an adult (60 kg), a dose within which exhibited no toxicity.

Examples of xanthine oxidase inhibitors suitable for use in the therapeutic compositions include, but are not limited to Allopurinol, hydroxyakalone, TEI-6720, carprofen, febuxostat, and y-700. U.S. Pat. No. 5,614,520 and U.S. Patent Pub. No. 2005/0090472 are cited for a non-limiting list of other examples. Representative RAS inihibitors include: captopril, cilazapril, enalapril, fosinopril, lisinopril, quinapril, ramapril, zofenopril, candesartan cilexetil, eprosartan, irbesartan, losartan, tasosartan, telmisartan, and valsartan, or pharmaceutically acceptable salts thereof.

In another embodiment, the subject invention pertains to a method of treating a stage of CKD in a diabetic or insulin insensitive subject comprising administering a therapeutically effective amount of a RAS inhibitor, or pharmaceutically acceptable salt thereof and coadministering a therapeutically effective amount of a conjunctive agent, or a pharmaceutically acceptable salt thereof, wherein said therapeutically effective amount of said conjunctive agent, or pharmaceutically acceptable salt thereof, comprises an amount sufficient to improve endothelial dysfunction and/or endothelial NO levels.

The term “coadministering” or “concurrent administration”, when used, for example with respect to administration of a conjunctive agent along with administration of a RAS inhibitor refers to administration of the RAS inhibitor and the conjunctive agent such that both can simultaneously achieve a physiological effect. The two agents, however, need not be administered together. In certain embodiments, administration of one agent can precede administration of the other, however, such coadministering typically results in both agents being simultaneously present in the body (e.g. in the plasma) at a significant fraction (e.g. 20% or greater, preferably 30% or 40% or greater, more preferably 50% or 60% or greater, most preferably 70% or 80% or 90% or greater) of their maximum serum concentration for any given dose.

The administration mode of the conjunctive formulation of the present invention is not particularly limited, provided that the compound of the present invention and the conjunctive drug are combined upon administration. Such an administration mode may, for example, be (1) an administration of a single formulation obtained by formulating a RAS inhibitor and conjunctive agent simultaneously, (2) a simultaneous administration via an identical route of two formulations obtained by formulating a RAS inhibitor and a conjunctive agent separately, (3) a sequential and intermittent administration via an identical route of two formulations obtained by formulating a RAS inhibitor and a conjunctive agent separately, (4) a simultaneous administration via different routes of two formulations obtained by formulating a RAS inhibitor and a conjunctive agent separately, (5) a sequential and intermittent administration via different routes of two formulations obtained by formulating RAS inhibitor and a conjunctive agent separately (for example, RAS inhibitor or its pharmaceutical composition followed by conjunctive agent or its pharmaceutical composition, or inverse order) and the like.

A diabetic nephropathy therapeutic composition of the present invention has a low toxicity, and thus a RAS inhibitor and conjunctive agent are mixed with a pharmacologically acceptable carrier in accordance with a method known per se to form a pharmaceutical composition, for example, a tablet (including sugar-coated and film-coated tablets), powder, granule, capsule (including soft capsule), solution, injection formulation, suppository, sustained release formulation and the like, which can safely be given orally or parenterally (e.g., topically, rectally, intravenously). An injection formulation may be given intravenously, intramuscularly, subcutaneously, into an organ, or directly into a lesion.

A pharmacologically acceptable carrier which may be employed for producing a conjunctive formulation of the present invention may, for example, be various organic and inorganic carrier materials employed customarily as pharmaceutical materials such as excipients, lubricants, binders and disintegrants in a solid formulation, solvents, dissolution aids, suspending agents, isotonicity imparting agents, bufferring agents and analgesic agents in a liquid formulation. Furthermore, other additives such as ordinary preservatives, antioxidants, colorants, sweeteners, adsorbents, wetting agents may also be added in suitable amounts.

An excipient may be, for example, lactose, sugar, D-mannitol, starch, corn starch, crystalline cellulose, light silicate anhydride and the like. A lubricant may, for example, be magnesium stearate, calcium stearate, talc, colloidal silica and the like.

A binder may be, for example, crystalline cellulose, sugar, D-mannitol, dextrin, hydroxypropyl cellulose, hydroxypropylmethyl cellulose, polyvinyl pyrrolidone, starch, sucrose, gelatin, methyl cellulose, sodium carboxymethyl cellulose and the like.

A disintegrant may be, for example, starch, carboxymethyl cellulose, calcium carboxymethyl cellulose, sodium carboxymethyl starch, L-hydroxypropyl cellulose and the like.

A solvent may be, for example, water for injection, alcohol, propylene glycol, macrogol, sesame oil, corn oil, olive oil and the like.

A dissolution aid may be, for example, polyethylene glycol, propylene glycol, D-mannitol, benzyl benzoate, ethanol, trisaminomethane, cholesterol, triethanolamine, sodium carbonate, sodium citrate and the like.

A suspending agent may be, for example, a surfactant such as stearyl triethanolamine, sodium lauryl sulfate, laurylaminopropionic acid, lecithin, benzalkonium chloride, benzethonium chloride, glycerin monostearate and the like; hydrophilic polymer such as polyvinyl alcohol, polyvinyl pyrrolidone, sodium carboxymethyl cellulose, methyl cellulose, hydroxymethyl cellulose, hydroxyethyl cellulose, hydroxypropyl cellulose and the like.

An isotonicity imparting agent may be, for example, glucose, D-sorbitol, sodium chloride, glycerin, D-mannitol and the like.

A buffering agent may be, for example, a buffer solution of a phosphate, acetate, carbonate, citrate and the like.

An analgesic may be, for example, benzyl alcohol.

A preservative may be, for example, a p-oxybenzoate, chlorobutanol, benzyl alcohol, phenethyl alcohol, dehydroacetic acid, sorbic acid and the like.

An antioxidant may, for example, be a sulfite, ascorbic acid, lipoic acid, α-tocopherol, EGCG and the like.

The ratio between a RAS inhibitor and a conjunctive agent in a conjunctive formulation of the present invention may be selected appropriately on the basis of the target and route. For example, the amount of a ACE inhibitor is usually about 0.01 to 100% by weight, typically about 0.1 to about 50% by weight, more specifically about 0.5 to about 20% by weight based on the entire formulation, although it may vary depending on the dosage form. The amount of nicorandil is usually about 0.01 to 100% by weight, typically about 0.1 to about 50% by weight, more specifically about 0.5 to about 20% by weight based on the entire formulation, although it may vary depending on the dosage form.

The amount of an additive such as a carrier contained in a conjunctive formulation of the present invention is usually about 1 to about 99.99% by weight, preferably about 10 to about 90% by weight based on the entire formulation, although it may vary depending on the dosage form.

Similar amounts may be employed also when a compound of the present invention and a conjunctive drug are formulated separately.

Such a formulation can be produced by a method known per se which is employed usually in a pharmaceutical process.

For example, a compound of the present invention and a conjunctive drug can be formulated with a dispersant (e.g., Tween 80 (ATLAS POWDER, USA), HCO60 (NIKKO CHEMICALS), polyethylene glycol, carboxymethyl cellulose, sodium alginate, hydroxypropylmethyl cellulose, dextrin), a stabilizer (e.g., ascorbic acid, sodium pyrosulfite), a surfactant (e.g., polysorbate 80, macrogol), a solubilizing agent (e.g., glycerin, ethanol), a buffering agent (phosphoric acid and its alkali metal salt, citric acid and its alkali metal salt and the like), an isotonizing agent (e.g., sodium chloride, potassium chloride, mannitol, sorbitol, glucose), a pH modifier (e.g., hydrochloric acid, sodium hydroxide), a preservative (e.g., ethyl p-oxybenzoate, benzoic acid, methylparabene, propylparabene, benzyl alcohol), a solubilizer (e.g., concentrated glycerin, meglumine), a solubilizing aid (e.g., propylene glycol, sugar), an analgesic (e.g., glucose, benzyl alcohol) into an aqueous formulation for injection, or dissolved, suspended or emulsified in a vegetable oil such as olive oil, sesame oil, cottonseed oil and corn oil and in a solubilizing aid such as propylene glycol to form an oily formulation, whereby producing an injection formulation.

In order to obtain an oral dosage form, a method known per se is employed to compact an inventive compound or a conjunctive drug for example with an excipient (e.g., lactose, sugar, starch), a disintegrant (e.g., starch, calcium carbonate), a binder (e.g., starch, gum Arabic, carboxymethyl cellulose, polyvinyl pyrrolidone, hydroxypropyl cellulose) or a glidant (e.g., talc, magnesium stearate, polyethylene glycol 6000) into a desired shape, which is then, if necessary, coated for the purpose of a taste masking, an enteric property or a sustained release performance by means of a coating method known per se, whereby obtaining an oral dosage form. Such a coating may, for example, be hydroxypropylmethyl cellulose, ethyl cellulose, hydroxymethyl cellulose, hydroxypropyl cellulose, polyoxyehtylene glycol, Tween 80, Pluronic F68, cellulose acetate phthalate, hydroxypropylmethyl cellulose phthalate, hydroxymethyl cellulose acetate succinate, Eudragit (Rohm, German, methacrylic/acrylic acid copolymer) and a colorant (e.g., iron oxide red, titanium dioxide). An oral dosage form may be an instantaneous release formulation or a sustained release formulation.

While the dose of an inventive conjunctive formulation may vary depending on the type of the inventive compound, the subject's age, body weight, condition, and the dosage form as well as administration mode and duration, for example, the daily dose in a patient experiencing symptoms of diabetes and/or insulin resistance and/or a stage of ckd (adult, body weight: about 60 kg) is about 0.01 to about 1000 mg/kg, preferably about 0.01 to about 100 mg/kg, more preferably about 0.1 to about 100 mg/kg, particularly about 0.1 to about 50 mg/kg, especially about 1.5 to about 30 mg/kg as an inventive compound, which is given intravenously at once or in several portions. It is a matter of course that the dose may vary depending on various factors as described above, and a less amount may sometimes be sufficient and an excessive amount should sometimes be required.

A conjunctive drug (e.g. nicorandil) may be employed in any amount within the range causing no problematic side effects. The daily dose of a conjunctive drug is not limited particularly and may vary depending on the severity of the disease, the subject's age, sex, body weight and susceptibility as well as time and interval of the administration and the characteristics, preparation, type and active ingredient of the pharmaceutical formulation, and the daily oral dose per kg body weight in a mammal is about 0.001 to 2000 mg, preferably about 0.01 to 500 mg, more preferably about 0.1 to about 100 mg as medicaments, which is given usually in 1 to 4 portions.

When an inventive conjunctive formulation is administered, it may be administered at the same time, but it is also possible that a conjunctive drug is first administered and then an inventive compound is administered, or that the inventive compound is first administered and then the conjunctive drug is administered. When such an intermittent administration is employed, the time interval may vary depending on the active ingredient administered, the dosage form and the administration mode, and for example, when the conjunctive drug is first administered, the inventive compound may be administered within 1 minute to 3 days, preferably 10 minutes to 1 day, more preferably 15 minutes to 1 hour after the administration of the conjunctive drug. When the inventive compound is first administered, for example, then the conjunctive drug may be administered within 1 minute to 1 day, preferably 10 minutes to 6 hours, more preferably 15 minutes to 1 hour after the administration of the inventive compound.

In a preferred administration mode, for example, about 0.001 to 200 mg/kg of a conjunctive drug formulated as an oral formulation is given orally as a daily dose, and, after about 15 minutes, about 0.005 to 100 mg/kg of an inventive compound formulated as an oral formulation is given orally as a daily dose.

Additional pharmacologically active agents may be delivered along with the primary active agents, RAS inhibitor and conjunctive agent. In one embodiment, such agents include, but are not limited to beta blockers, statins, and aspirin. Suitable statins are well known to those of skill in the art. Such statins include, but are not limited to atorvastatin (LIPITOR®, Pfizer), simvastatin (ZOCOR®, Merck0, pravastatin (PRAVACHOL®, Bristol-Myers Squibb), fluvastatin (LESCOL®, Novartis), lovastatin (MEVACOR®, Merck), rosuvastatin (Crestor®, Astra Zeneca), and Pitavastatin (Sankyo), and the like. Suitable beta blockers include, but are not limited to cardioselective (selective beta 1 blockers), e.g., acebutolol, atenolol, betaxolol, bisoprolol, metoprolol, and the like. Suitable non-selective blockers (block beta 1 and beta 2 equally) include, but are not limited to carteolol, nadolol, penbutolol, pindolol, propranolol, timolol, labetalol,and the like.

The invention is further supported by the following Examples, which are not intended to restrict the invention.

A value indicated for a solvent mixture is a volume ratio of each solvent, unless otherwise specified. A % is a % by weight, unless otherwise specified. A ratio of the elution solvent in a chromatography on a silica gel is a volume ratio, unless otherwise specified. Room temperature (ambient temperature) employed here usually means a temperature from about 20 to about 30° C.

Examples Evidencing Therapeutic Value of Conjunctive Therapy for Diabetic Nephropathy and/or Metabolic Syndrome related consequences

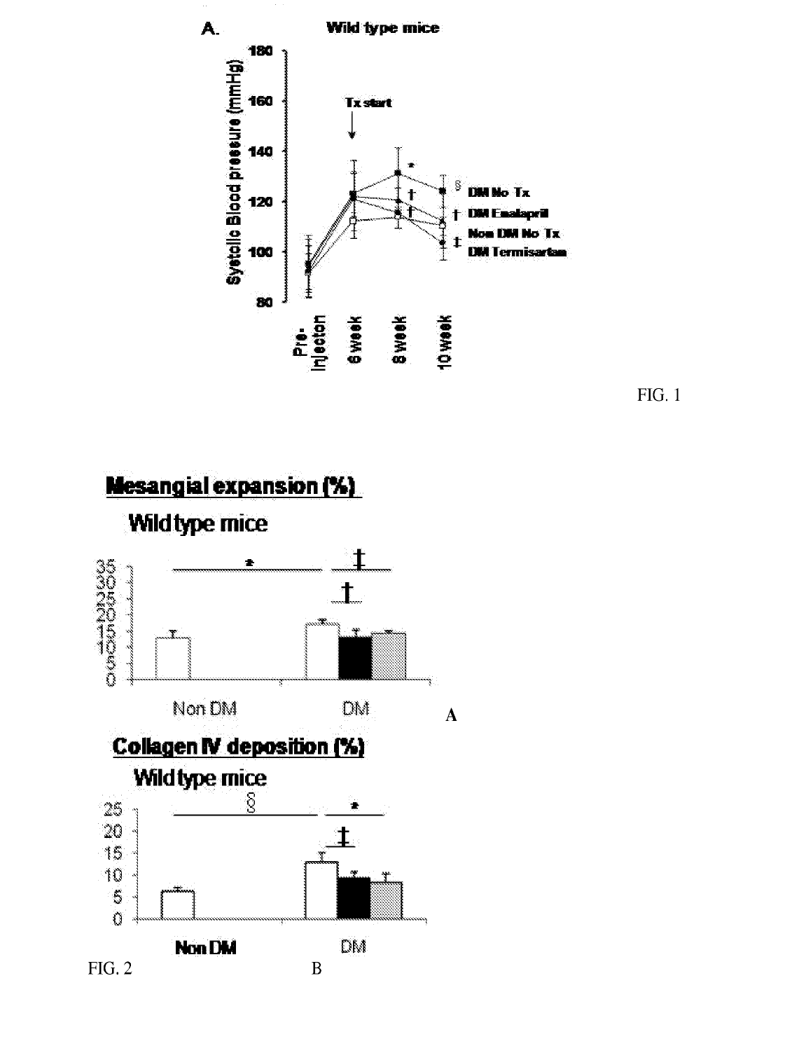

FIG. 1 is a graph showing that ACE inhibitors are effective in lowering blood pressure in wild type diabetic (DM) mice. Shown in FIG. 1 is the effect of no treatment (No TX), the ACE inhibitor enalapril (DM enalapril), and the ARB telmisartan (DM telmisartan). Both the ACE inhibitor and ARB can significantly lower blood pressure (BP) in wild type diabetic mice. FIG. 2 demonstrates that administration of ACE inhibitors (enalapril) and ARBs (telmisartan) also improve mesangial expansion (A) and glomerular type IV collagen deposition (B) in wild type diabetic (DM) mice. These are considered early changes of diabetic nephropathy.

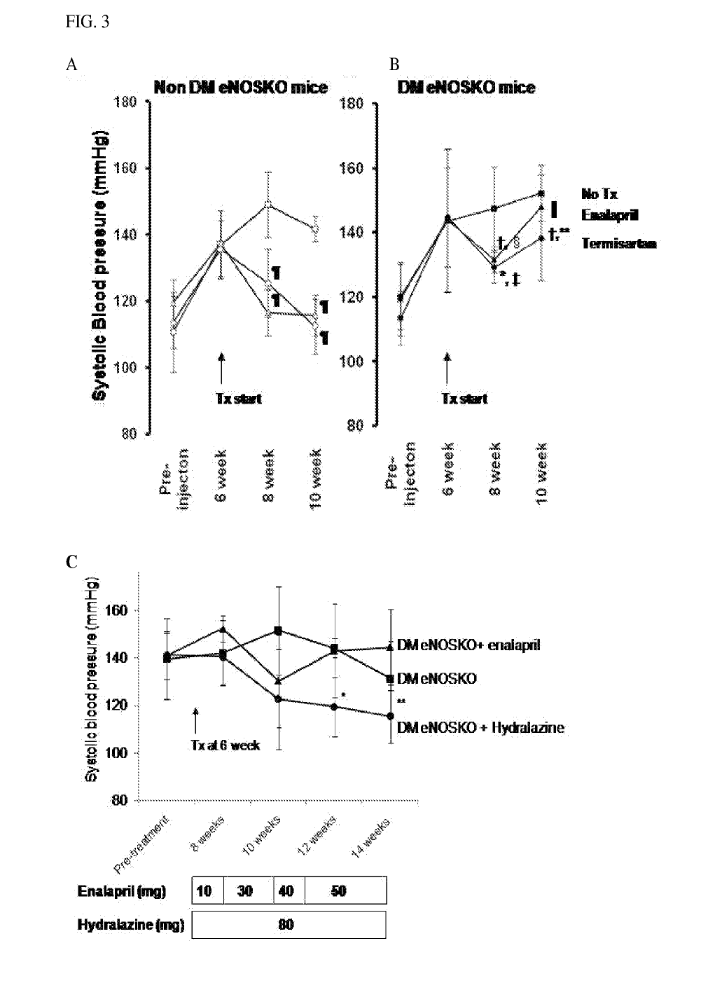

In contrast, when ACE inhibitors or ARBs were administered to diabetic mice lacking the ability to produce endothelial nitric oxide, the surprising and novel finding was that these agents were minimally effective (FIGS. 3-5). FIG. 3 is a graph showing that administration of ACE inhibitors and ARBs are Less Effective at Lowering Blood Pressure in Diabetic Mice lacking Endothelial NO. ACE inhibitors and ARBs are effective at lowering BP in nondiabetic mice lacking endothelial nitric oxide synthesis (eNOSKO) (FIG. 3A). In contrast, while an initial reduction in blood pressure was observed at 8 weeks in diabetic eNOSKO mice, this was not maintained (FIG. 3C. This contrasts with other antihypertensive agents such as hydralazine which are very effective¹⁸. The lack of effect is also not due to dose, as we found that even doses of enalapril 50 mg/kg did not lower BP in diabetic mice lacking endothelial nitric oxide synthesis (FIG. 3C).

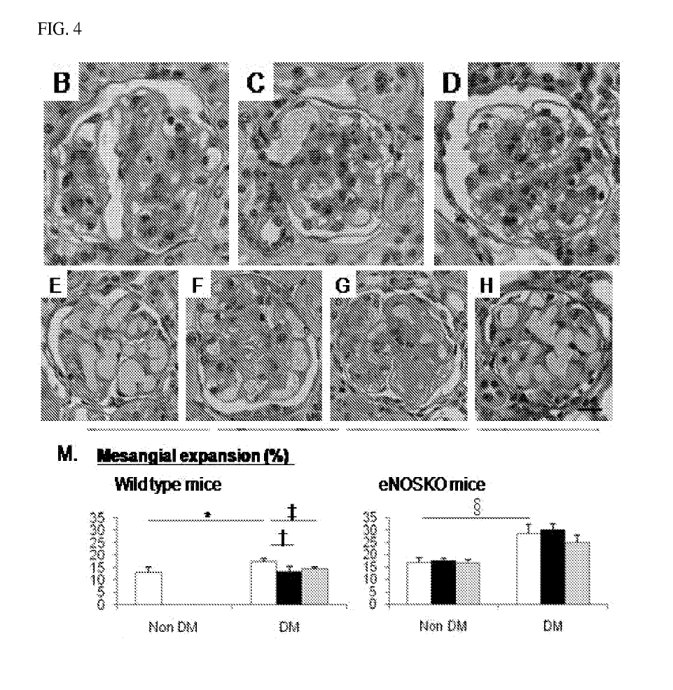

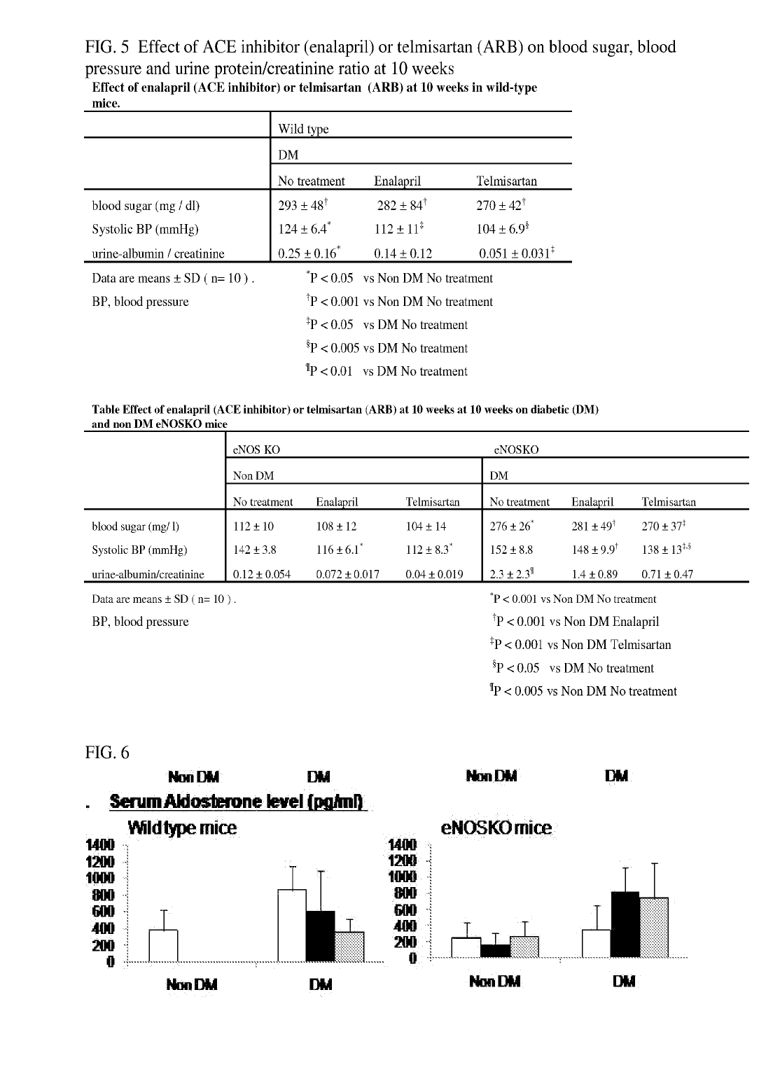

FIG. 4 shows histological slides revealing that ACE inhibitors and ARBs are not Effective at Preventing Diabetic Nephropathy in eNOSKO mice. DM caused mesangial expansion in DM eNOSKO mice (b), which was not prevented by enalapril (c) or telmisartan (d). DM eNOSKO mice also exhibited mesangiolysis with glomerular capillary microaneurysms (e) and with augmented deposition of extracellular matrix (f). Neither enalapril (g) nor telmisartan (h) treatment prevented these advanced lesions. Bar: 20 μm. Quantitative analysis of mesangial expansion (M) confirmed a beneficial effect of ACE inhibitor (black bar) and ARB (grey bar) in wild type diabetic mice but not diabetic eNOSKO mice. Bar: 20 μm. White bar=no treatment; Black bar=enalapril; Gray bar=telmisartan. FIG. 5 pertains to a table that provides results of the effect of ACE inhibitors and ARBs on blood pressure and renal function in diabetic nephropathy in eNOSKO mice. ACE inhibitors and ARBs are effective at lowering blood pressure and proteinuria in wild type diabetic mice, and are also effective at lowering blood pressure in nondiabetic eNOSKO mice. However, ACE inhibitors are ineffective and ARAB are only minimally effective at lowering blood pressure and do not significantly reduce proteinuria in diabetic eNOSKO mice at 10 weeks.

These data provide evidence that there is a specific interaction between a lack of endothelial NO and diabetes with regard to ACE and ARB efficacy—this would not be predicted based on studies of diabetes or eNOS deficiency alone in which these latter agents are effective.

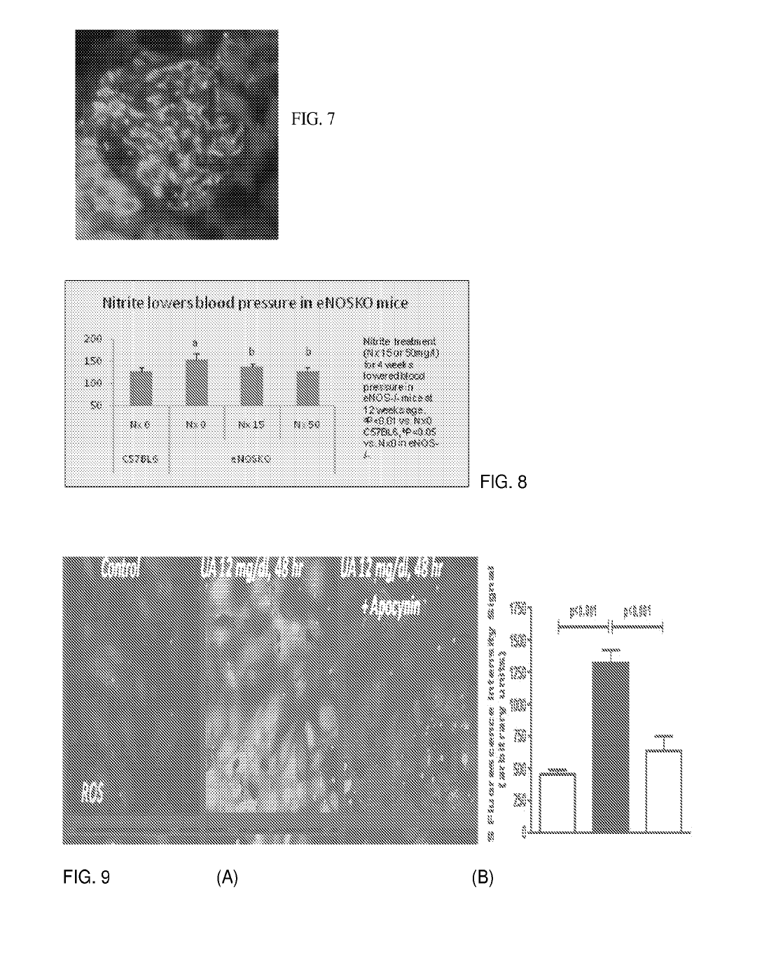

The inability of ACE inhibitors and ARBs to protect against diabetic nephropathy in the presence of endothelial dysfunction (lack of endothelial NO) might be accounted by the presence of an aldosterone breakthrough with suppression of the endogenous renin angiotensin system. Consistent with this observation, the inventors found that serum aldosterone was suppressed by ACE inhibitor and ARB treatment in wild type diabetic mice, but not in diabetic eNOSKO mice (FIG. 6). In addition, in diabetic eNOS KO mice treated with an ACE inhibitor, aldosterone was identified in glomeruli (FIG. 7).

FIG. 6 represent graphs showing that serum aldosterone is not suppressed by ACE inhibitor or ARB treatment in diabetic eNOS KO mice but is suppressed in diabetic wild type mice. FIG. 7 is a immunohistograph showing that diabetic eNOSKO mice have an increase in aldosterone in their glomeruli. Diabetes was induced with streptozotocin in eNOSKO mice, which were treated with enalapril (10 mg/kg BW/day; black bar) or telmisartan (2 mg/kg BW/day; gray bar) for 4 weeks (from at 6 weeks to 10 weeks) ²². Immunohistochemistry for aldosterone in the kidney of diabetic eNOSKO with enalapril treatment was examined by using a rabbit polyclonal anti-aldosterone antibody (Thermo Fisher Scientific, Rockford, Ill.). As shown in the figure, aldosterone was present in glomerulus in this model. While this data does not necessarily mean that aldosterone is synthesize in glomerulus, aldosterone can be produced in vessels, in particular endothelial cellc ^{23, 24}. The positive green staining represents the presence of aldosterone.

These studies provide evidence that a lack, or severe reduction, in endothelial NO renders ACE inhibitors and ARBs ineffective in the treatment of diabetic nephropathy, and that the mechanism is likely via an aldosterone breakthrough. They also demonstrate that the lack of responsiveness of ACE inhibitors and of ARBs is not expected based on the presence of diabetes alone, or endothelial dysfunction (lack of endothelial NO) alone, but is specific to their interaction. Endothelial dysfunction is common in diabetes despite the use of ACE inhibitors²⁵⁻²⁷, but no one has suggested that there is a specific interaction of endothelial dysfunction with diabetes that results in ACE inhibitor responsiveness. Since the only abnormality in the eNOSKO mouse (compared to wild type) is the lack of endothelial NO, these data suggest that replacement of nitric oxide should reverse the defect in this mouse.

In this regard, eNOSKO mice have higher baseline blood pressure than their wild type counterpart. As shown in FIG. 8, if nitrites (an NO donor) are provided to an eNOSKO mouse, we can reduce blood pressure to the same level as a wild type mouse. FIG. 8 represents a graph showing that supply of a nitric oxide mimetic (Nitrite) can correct the blood pressure abnormality in the eNOSKO mouse. Since nitrite is a metabolite of NO, and can be converted to NO in the body (reviewed in ²⁸), sodium nitrite was tested to see if it could prevent hypertension in eNOSKO mice. Treatment with nitrite for 4 weeks (50 mg/l drinking water) reduced blood pressure in eNOSKO mice similar to that observed in wild type mice.

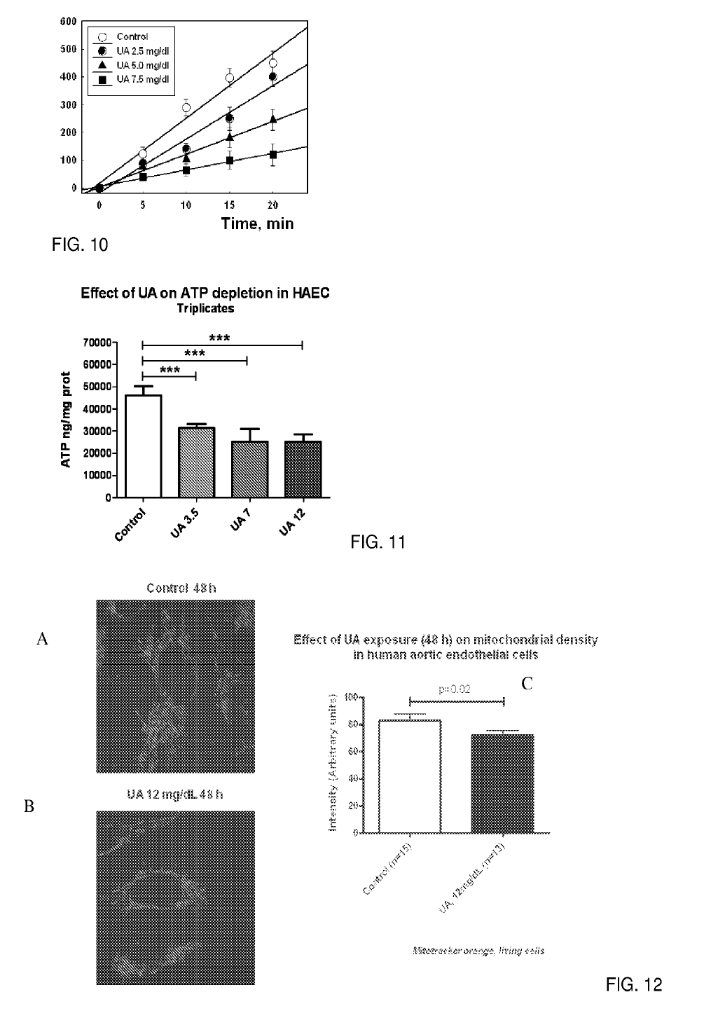

Given the key role of endothelial dysfunction in causing unresponsiveness of diabetic nephropathy to ACE inhibitors and ARBs, therapies increasing endothelial NO levels will be beneficial if combined with ACE inhibitors in diabetic nephropathy. The major cause of endothelial NO reduction in diabetic nephropathy is oxidative stress²⁹, and hence antioxidants should have a unique benefit when combined with ACE inhibitors or ARBs in diabetic nephropathy. Another source of oxidative stress is uric acid; for which we have shown can both stimulate oxidative stress in endothelial cells (FIG. 9) and also cause a reduction in endothelial nitric oxide^{30, 31} (FIG. 10). Therefore, agents that lower uric acid, such as xanthine oxidase inhibitors (febuxostat and allopurinol) and uricosuric agents (such as probenecid, benziodarone and benzbromarone) should also be beneficial in diabetic nephropathy when combined with ACE inhibitors or ARBs, particularly in subjects with uric acid levels >6 mg/dl in which endothelial function is known to be commonly impaired³². Indeed, it has recently been found that uric acid is a powerful predictor of overt diabetic nephropathy in subjects with type 1 diabetes³³.

One of the key mechanisms by which uric acid acts to cause endothelial dysfunction is to cause a loss of mitochondria and mitochondria DNA, resulting in depletion of ATP stores necessary for endothelial function (FIGS. 11 and 12). FIG. 11 shows that uric acid reduces ATP levels in human aortic endothelial cells. Morevoer, FIG. 12 shows that uric acid reduces mitochondria number in human aortic endothelial cells.

The observation that an NO donor, such as nicorandil, can prevent mitochondrial loss in response to oxidative stress provides a mechanism by which NO may be able to improve endothelial function and enhance ACE responsiveness in the diabetic subject. Accordingly, it has been realized that conditions associated with NO deficiency and diabetic kidney disease, such as elevated uric acid, have been shown to be associated with loss of mitochondria.

References

1. Lewis E J, Hunsicker L G, Bain R P, Rohde R D: The effect of angiotensin-converting-enzyme inhibition on diabetic nephropathy. The Collaborative Study Group, N Engl J Med 1993, 329:1456-1462

2. Brenner B M, Cooper M E, de Zeeuw D, Keane W F, Mitch W E, Parving H H, Remuzzi G, Snapinn S M, Zhang Z, Shahinfar S: Effects of losartan on renal and cardiovascular outcomes in patients with type 2 diabetes and nephropathy, N Engl J Med 2001, 345:861-869

3. Carter B L, Malone D C, Ellis S L, Dombrowski R C: Antihypertensive Drug Utilization in Hypertensive Veterans With Complex Medication Profiles, J Clin Hypertens (Greenwich) 2000, 2:172-180

4. Scarsi K K, Bjornson D C: The use of ACE inhibitors as renoprotective agents in Medicaid patients with diabetes, Ann Pharmacother 2000, 34:1002-1006

5. Mauer M, Zinman B, Gardiner R, Suissa S, Sinaiko A, Strand T, Drummond K, Donnelly S, Goodyer P, Gubler M C, Klein R: Renal and retinal effects of enalapril and losartan in type 1 diabetes, N Engl J Med 2009, 361:40-51

6. Perkins B A, Ficociello L H, Silva K H, Finkelstein D M, Warram J H, Krolewski A S: Regression of microalbuminuria in type 1 diabetes, N Engl J Med 2003, 348:2285-2293

7. Suissa S, Hutchinson T, Brophy J M, Kezouh A: ACE-inhibitor use and the long-term risk of renal failure in diabetes, Kidney Int 2006, 69:913-919

8. Ruggenenti P, Mosconi L, Sangalli F, Casiraghi F, Gambara V, Remuzzi G, Remuzzi A: Glomerular size-selective dysfunction in NIDDM is not ameliorated by ACE inhibition or by calcium channel blockade, Kidney Int 1999, 55:984-994

9. Zatz R, Dunn B R, Meyer T W, Anderson S, Rennke H G, Brenner B M: Prevention of diabetic glomerulopathy by pharmacological amelioration of glomerular capillary hypertension, J Clin Invest 1986, 77:1925-1930

10. Zatz R, Meyer T W, Rennke H G, Brenner B M: Predominance of hemodynamic rather than metabolic factors in the pathogenesis of diabetic glomerulopathy, Proc Natl Acad Sci U S A 1985, 82:5963-5967

11. Bomback A S, Klemmer P J: The incidence and implications of aldosterone breakthrough, Nat Clin Pract Nephrol 2007, 3:486-492

12. Hollenberg N K: Aldosterone in the development and progression of renal injury, Kidney Int 2004, 66:1-9

13. Schjoedt K J, Andersen S, Rossing P, Tarnow L, Parving H H: Aldosterone escape during blockade of the renin-angiotensin-aldosterone system in diabetic nephropathy is associated with enhanced decline in glomerular filtration rate, Diabetologia 2004, 47:1936-1939

14. Saklayen M G, Gyebi L K, Tasosa J, Yap J: Effects of additive therapy with spironolactone on proteinuria in diabetic patients already on ACE inhibitor or ARB therapy: results of a randomized, placebo-controlled, double-blind, crossover trial, J Investig Med 2008, 56:714-719

15. Breyer M D, Bottinger E, Brosius F C, 3rd, Coffman T M, Harris R C, Heilig C W, Sharma K: Mouse models of diabetic nephropathy, J Am Soc Nephrol 2005, 16:27-45

16. Nakagawa T, Sato W, Glushakova O, Heinig M, Clarke T, Campbell-Thompson M, Yuzawa Y, Atkinson M A, Johnson R J, Croker B: Diabetic endothelial nitric oxide synthase knockout mice develop advanced diabetic nephropathy, J Am Soc Nephrol 2007, 18:539-550

17. Sato W, Kosugi T, Zhang L, Roncal C A, Heinig M, Campbell-Thompson M, Yuzawa Y, Atkinson M A, Grant M B, Croker B P, Nakagawa T: The pivotal role of VEGF on glomerular macrophage infiltration in advanced diabetic nephropathy, Lab Invest 2008,88:949-961

18. Kosugi T, Heinig M, Nakayama T, Connor T, Yuzawa Y, Li Q, Hauswirth W W, Grant M B, Croker B P, Campbell-Thompson M, Zhang L, Atkinson M A, Segal M S, Nakagawa T: Lowering blood pressure blocks mesangiolysis and mesangial nodules, but not tubulointerstitial injury, in diabetic eNOS knockout mice, Am J Pathol 2009, 174:1221-1229

19. Kosugi T, Nakayama T, Heinig M, Matsuo S, Nakagawa T: Effect of Renin-Angiotensin Blockade in Advanced Diabetic Nephropathy in eNOS Knockout Mice: Superior Protection with Aldosterone Receptor Antagonist, Am J Pathol in press,

20. Zhao H J, Wang S, Cheng H, Zhang M Z, Takahashi T, Fogo A B, Breyer M D, Harris R C: Endothelial nitric oxide synthase deficiency produces accelerated nephropathy in diabetic mice, J Am Soc Nephrol 2006, 17:2664-2669

21. Nakagawa T, Sato W, Glushakova O, Heinig M, Clarke T, Campbell-Thompson M, Yuzawa Y, Atkinson M, Johnson R J, Croker B: Diabetic eNOS knockout mice develop advanced diabetic nephropathy, J Am Soc Nephrol 2007, 18:539-550

22. Kosugi T, Heinig M, Nakayama T, Matsuo S, Nakagawa T: Less benefit of Renin-Angiotensin Blockade in Advanced Diabetic Nephropathy in eNOS Knockout Mice: Superior Protection of Aldosterone Receptor Antagonist, Am J Pathol In press,

23. Oberleithner H, Ludwig T, Riethmuller C, Hillebrand U, Albermann L, Schafer C, Shahin V, Schillers H: Human endothelium: target for aldosterone, Hypertension 2004, 43:952-956

24. Takeda Y, Miyamori I, Yoneda T, Iki K, Hatakeyama H, Blair I A, Hsieh F Y, Takeda R: Production of aldosterone in isolated rat blood vessels, Hypertension 1995, 25:170-173

25. Jawa A, Nachimuthu S, Pendergrass M, Asnani S, Fonseca V: Impaired vascular reactivity in African-American patients with type 2 diabetes mellitus and microalbuminuria or proteinuria despite angiotensin-converting enzyme inhibitor therapy, J Clin Endocrinol Metab 2006, 91:31-35

26. Papaioannou G I, Seip R L, Grey N J, Katten D, Taylor A, Inzucchi S E, Young L H, Chyun D A, Davey J A, Wackers F J, Iskandrian A E, Ratner R E, Robinson E C, Carolan S, Engel S, Heller G V: Brachial artery reactivity in asymptomatic patients with type 2 diabetes mellitus and microalbuminuria (from the Detection of lschemia in Asymptomatic Diabetics-brachial artery reactivity study), Am J Cardiol 2004, 94:294-299

27. Chan W B, Chan N N, Lai C W, So W Y, Lo M K, Lee K F, Chow C C, Metreweli C, Chan J C: Vascular defect beyond the endothelium in type II diabetic patients with overt nephropathy and moderate renal insufficiency, Kidney Int 2006, 70:711-716

28. Bryan N S: Nitrite in nitric oxide biology: cause or consequence? A systems-based review, Free Radic Biol Med 2006, 41:691-701

29. Munzel T, Sinning C, Post F, Warnholtz A, Schulz E: Pathophysiology, diagnosis and prognostic implications of endothelial dysfunction, Ann Med 2008, 40:180-196

30. Khosla U M, Zharikov S, Finch J L, Nakagawa T, Roncal C, Mu W, Krotova K, Block E R, Prabhakar S, Johnson RJ: Hyperuricemia induces endothelial dysfunction, Kidney Int 2005, 67:1739-1742

31. Zharikov S I, Krotova K, Hu H, Baylis C, Johnson R J, Block E R, Patel J M: Uric Acid Decreases No Production and Increases Arginase Activity in Cultured Pulmonary Artery Endothelial Cells, Am J Physiol Cell Physiol 2008,

32. Zoccali C, Maio R, Mallamaci F, Sesti G, Perticone F: Uric acid and endothelial dysfunction in essential hypertension, J Am Soc Nephrol 2006, 17:1466-1471

33. Hovind P, Rossing P, Tarnow L, Johnson R J, Parving H H: Serum uric acid as a predictor for development of diabetic nephropathy in type 1 diabetes: an inception cohort study, Diabetes 2009, 58:1668-1671

1. A therapeutic composition useful for treating chronic kidney disease in a patient exhibiting at least one symptom of a metabolic imbalance, said composition comprising a RAS inhibitor and a NO mimetic.

2. The composition of claim 1, wherein said NO mimetic is nicorandil.

3. The composition of claim 1, wherein said at least one symptom of a metabolic imbalance comprises elevated waist circumference, elevated triglycerides, reduced HDL cholesterol, elevated blood pressure or elevated fasting glucose.

4. The composition of claim 1, wherein said at least one symptom of a metabolic imbalance is elevated blood pressure and elevated fasting glucose.

5. The composition of claim 1 formulated in solid dosage form comprising a powder, granule, tablet, pill, or capsule.

6. The composition of claim 1 formulated in a liquid suspension or solution.

7. The composition of claim 1, wherein said chronic kidney disease is stage 1, stage 2 or stage 3 CKD.

8. A method of treating chronic kidney disease in a patient exhibiting at least one symptom of a metabolic imbalance, the method comprising coadministering a therapeutically effective amount of a RAS inhibitor and a conjunctive agent.

9. The method of claim 8, wherein said RAS inhibitor is an ACE inhibitor or angiotensin receptor blocker.

10. The method of claim 8, wherein said conjunctive agent is a uric acid lowering agent.

11. The method of claim 10, wherein said uric acid lowering agent is a xanthine oxidase inhibitor.

12. The method of claim 11, wherein said xanthine oxidase inhibitor is allopurinol or febuxostat.

13. The method of claim 8, wherein said conjunctive agent is a NO mimetic.

14. The method of claim 13, wherein said NO mimetic is L-arginine or nicorandil.

15. The method of claim 8, wherein said RAS inhibitor and said conjunctive agent are administered orally.

16. The method of claim 8, wherein said RAS inhibitor and said conjunctive agent are administered parenterally.

17. The method of claim 8, wherein said RAS inhibitor and said conjunctive agent are administered together in a solid dosage form comprising a powder, granule, tablet, pill, or capsule.

18. The method of claim 8, wherein said RAS inhibitor is administered according to a first mode of administration and said conjunctive agent is administered according to a second mode of administration different from said first mode.

19. The method of claim 8, wherein said at least one symptom of a metabolic imbalance comprises elevated waist circumference, elevated triglycerides, reduced HDL cholesterol, elevated blood pressure or elevated fasting glucose.

20. The method of claim 8, wherein said at least one symptom of a metabolic imbalance is elevated fasting glucose.

21. The method of claim 8, wherein said chronic kidney disease is stage 1, stage 2, stage 3, stage 4, stage 5 or stage 6 CKD.

22. The method of claim 8, wherein said chronic kidney disease is stage 1, stage 2, stage 3 or stage 4.

23. A method of treating chronic kidney disease in a patient exhibiting at least one symptom of a metabolic imbalance, said method comprising administering a therapeutically effective amount of nicorandil.

24. The method of claim 23, further comprising coadministering nicorandil with an ACE inhibitor and/or an angiotensin receptor blocker.

25. A composition useful for treating diabetic nephropathy comprising a therapeutically effective amount of a RAS inhibitor and a uric acid lowering agent or antioxidant, or a combination of a uric acid lowering agent and antioxidant.
